(19) United States
(12) Patent Application Publication
(10) Pub. No.: US 2008/0038843 A1
(43) Pub. Date: Feb. 14, 2008

(54) METHOD OF DIAGNOSING CELIAC DISEASE

(75) Inventors: Alessio Fasano, West Friendship, MD (US); Deborah Kryszak, Columbia, MD (US); Julio C. Bai, Buenos Aires (AR)

Correspondence Address: Connolly Bove Lodge Hutz, LLP;(FOR ALBA THERAPEUTICS), P.O. BOX 2207, WILMINGTON, DE, 19899-2207, US

(73) Assignees: University of Maryland - Baltimore, Baltimore, MD (US); ALBA Therapeutics Corp., Baltimore, MD (US)

(21) Appl. No.: 11/835,062

(22) Filed: Aug. 7, 2007

Related U.S. Application Data
(60) Provisional application No. 60/836,988, filed on Aug. 11, 2006.

(51) Int. Cl. G01N 33/564; G01N 33/00

(52) U.S. Cl. 436/506; 436/86

(57) ABSTRACT

The present invention provides materials and methods to diagnose celiac disease. In some embodiments, the present invention provides a method of diagnosing celiac disease by measuring the serum zonulin level of a subject.

### CROSS REFERENCE TO RELATED APPLICATIONS

This application claims priority to U.S. provisional patent application Ser. No. 60/836,988, filed Aug. 11, 2006, the entire contents of which are specifically incorporated herein by reference.

### FIELD OF THE INVENTION

The present invention relates to the field of diagnostics.

### BACKGROUND OF THE INVENTION

Mammalian epithelia contain structures referred to as zonula occludens (ZO) also referred to as tight junctions (TJs). These structures regulate the passage of materials through the epithelia by controlling access to the space between the epithelial cells (the paracellular pathway). To meet the many diverse physiological and pathological challenges to which epithelia are subjected, the tight junctions or zonula occludens must be capable of rapid, physiologic, reversible, transient, energy dependent, and coordinated responses that require the presence of a complex regulatory system. Examples of epithelia containing tight junctions include, but are not limited to, the intestines (particularly the small intestine), and the blood brain barrier.

In the absence of stimuli, the tight junctions are closed restricting access to the paracellular pathway. In the presence of stimuli, the tight junctions are reversibly opened. In U.S. Pat. Nos. 5,945,510 and 5,948,629, novel mammalian proteins that function as the physiological modulator of mammalian tight junctions, have been identified and purified. These mammalian proteins, referred to as “zonulin,” function as the physiological effector of mammalian tight junctions. Certain bacteria have been shown to have toxins that stimulate the opening of tight junctions. Vibrio cholerae infected with the filamentous bacteriophage CTXφ, produces a toxin (zonula occludens toxin, ZOT) that has been shown to cause opening of tight junctions. It has been shown that 6 His-ΔG, an N-terminal deletion of ZOT in which the first 264 amino acids have been deleted and replaced with a six histidine purification tag, retains the ability to open tight junctions.

Intestinal tight junction dysfunction occurs in auto-immune diseases and in a variety of clinical conditions affecting the gastrointestinal tract, including food allergies, enteric infections, malabsorption syndromes such as celiac disease, and inflammatory bowel diseases. Healthy, mature gut mucosa with its intact tight junction serves as the main barrier to the passage of macromolecules. During the healthy state, small quantities of immunologically active proteins cross the gut host barrier. When the integrity of the tight junction system is compromised, as with prematurity or after exposure to radiation, chemotherapy, and/or toxins, a deleterious response to environmental antigens (including autoimmune diseases and food allergies) can occur.

Celiac disease (CD) is a condition in which ingestion of gluten (a protein found in wheat, rye, and barley) results in an abnormal increase in intestinal tight junction permeability. It is well known that celiac disease can be associated with other autoimmune diseases (AD). Recently, a positive correlation between anti-actin auto-antibodies and severity of intestinal villus damage in celiac disease has been shown (Carroccio, et al., Clinical Chemistry 51:917-920, 2005). Currently, the only treatment available for celiac disease is a gluten-free diet (GFD).

Presently, the only definite test for celiac disease is an upper GI endoscopy (EGD) with an intestinal biopsy. This procedure is highly invasive and entails the risk of placing the subject under anesthesia. It would be useful to have a less invasive and risky method of determining if a subject had celiac disease. This need and others are met by the present invention.

### SUMMARY OF THE INVENTION

The present invention provides materials and methods for diagnosing celiac disease in a subject. Such methods may comprise obtaining a sample from the subject and determining zonulin and at least one auto-antibody in the sample. Typically, the detection of both zonulin and the auto-antibody is predictive of the presence of celiac disease. Typically, failure to detect zonulin coupled to failure to detect the auto-antibody is predictive of the absence of celiac disease. Any type of sample known to those skilled in the art may be obtained and analyzed for the presence or absence of zonulin and/or one or more auto-antibodies. In one embodiment, the sample obtained from the subject may be a blood sample. In some embodiments, serum may be isolated from the blood sample and zonulin and at least one auto-antibody may be determined in the serum. In one specific embodiment, methods of the invention may comprise obtaining a sample from a subject and determining zonulin and anti-actin auto-antibody in the sample. In embodiments of this type, detecting the presence of both zonulin and anti-actin auto-antibody is predictive of celiac disease and failing to detect zonulin and failing to detect anti-actin auto-antibody is predictive of the absence of celiac disease. In another specific embodiment, methods of the invention may comprise obtaining a sample from a subject, determining zonulin, anti-actin auto-antibody and anti-transglutaminase auto-antibody, wherein detecting the presence of all three is predictive of celiac disease and failing to detect any of the three is predictive of the absence of celiac disease.

Practice of some embodiments of the invention may require the determination of zonulin. Determining zonulin includes both detecting the presence or absence of zonulin in a sample as well as measuring the concentration of zonulin in a sample. In some embodiments of the invention, the concentration of zonulin may be measured while in other embodiments of the invention the presence or absence of zonulin may be assayed. Zonulin may be determined using any technique known to those of skill in the art. In some embodiments, determining zonulin may comprise contacting the sample with a first antibody that binds to zonulin under binding conditions, contacting the bound sample with a second antibody that binds zonulin under binding conditions, and detecting the presence of bound second antibody. Any first and second antibodies may used in the practice of the invention so long as they bind to both bind to zonulin with sufficient affinity to permit detection. In some embodiments, at least one antibody may be raised against a protein comprising a fragment of zonula occludens toxin. In one particular embodiment, the first antibody may be raised against a protein comprising a fragment of zonula occludens toxin, for example, the ΔG fragment of zonula occludens toxin. In some embodiments, at least one antibody may be raised against a protein comprising zonula occludens toxin. In a particular embodiment, the second antibody may be raised against a protein comprising zonula occludens toxin. Typically, the second antibody may comprise a detectable moiety, for example, biotin.

The present invention provides kits for diagnosing celiac disease. In some embodiments, kits of the invention may comprise means for detecting zonulin and means for detecting at least one auto-antibody. Means for detecting zonulin may comprise one or more containers containing one or more antibodies. For example, a kit of the invention may comprise a first container containing a first antibody and a second container containing a second antibody. Any antibody capable of binding zonulin may be used as a first or second antibody. Typically, the first and second antibodies bind at different regions of zonulin such that both a first and a second antibody may be bound to zonulin at the same time. In some embodiments, kits of the invention may comprise one or more containers containing antibodies wherein at least one antibody was raised against a protein comprising a fragment of zonula occludens toxin. In some embodiments, kits of the invention may comprise one or more containers containing antibodies wherein the first antibody was raised against a protein comprising a fragment of zonula occludens toxin. In some embodiments, kits of the invention may comprise one or more containers containing antibodies wherein the first antibody was raised against a protein comprising AG fragment of zonula occludens toxin. In some embodiments, kits of the invention may comprise one or more containers containing antibodies wherein at least one antibody was raised against a protein comprising zonula occludens toxin. In some embodiments, kits of the invention may comprise one or more containers containing antibodies wherein the second antibody was raised against a protein comprising zonula occludens toxin. In some embodiments, kits of the invention may comprise one or more containers containing antibodies wherein the second antibody comprises a detectable moiety, for example, biotin. Kits of the invention may also comprise one or more additional containers containing one or more additional reagents. Additional reagents include salts, buffers, metal ions, enzyme substrates and the like. In some embodiments, kits of the invention may comprise a container containing ΔG fragment of zonula occludens toxin. In some embodiments, kits of the invention may comprise a container containing zonulin. Kits of the invention may comprise one or more protein binding partners of auto-antibodies. For example, kits of the invention may comprise one or more containers containing one or more of human transglutaminase (tTG) and/or human actin.

### DETAILED DESCRIPTION OF THE INVENTION

As used herein a subject is any animal, e.g., mammal, that receives a treatment. Subjects include, but are not limited to, humans.

As used herein, antibodies are named using the prefix anti- followed by the antigen to which the antibody binds. Thus, anti-actin is an antibody that binds to actin. Auto-antibodies are antibodies produced by a subject that recognize self antigens. Thus, anti-actin auto-antibody is an antibody produced by a subject that binds to actin produced by the same subject.

### EXAMPLE 1

Sixty-eight consecutive patients (average age 38 yrs, range 18-80) referred by the Small Bowel disorders clinic to EGD and intestinal biopsy because of suspicion of intestinal disorders (chronic diarrhea, weight loss, abdominal distention, recurrent abdominal pain, and/or anemia) were enrolled between August 2004 and July 2005 (Group A). During the same period, a random sample of patients (n=98, average age 47 yrs, range 23-73) undergoing to an EGD for symptoms or disorders not related to CD was also enrolled (Group B). All duodenal biopsies (BX) were blindly scored using the modified Marsh criteria by pathologists. At the time of the procedure, a serum sample was also obtained to check for serum anti-actin antibodies (AAA), anti-tissue transglutaminase antibodies (TTG), and zonulin levels. Results of the assays are shown in the following tables

TABLE 1
Group A CD positive by biopsy

| AAA | tTG | Zonulin | BX | Number |
|---|---|---|---|---|
| + | + | + | + | 26 |
| | | + | + | 3 |
| | + | + | + | 4 |
| | + | | + | 0 |
| + | + | | + | 0 |
| + | | + | + | 0 |
| | | | + | 0 |
| | | | + | 0 |
| | | | | 33 |

TABLE 2
Group A no CD by biopsy

| AAA | tTG | Zonulin | BX | Number |
|---|---|---|---|---|
| + | + | + | neg | 0 |
| | | + | neg | 21 |
| | + | + | neg | 1 |
| | + | | neg | 0 |
| + | + | | neg | 0 |
| + | | + | neg | 2 |
| + | | | neg | 0 |
| | | | neg | 11 |
| | | | | 35 |

TABLE 3
Group B CD positive by biopsy

| AAA | tTG | Zonulin | BX | Number |
|---|---|---|---|---|
| + | + | + | + | 1 |
| | | + | + | 0 |
| | + | + | + | 0 |
| | + | | + | 0 |
| + | + | | + | 0 |
| + | | + | + | 0 |
| | | | + | 0 |
| | | | + | 0 |
| | | | | 1 |

TABLE 4
Group B No CD by biopsy

| AAA | tTG | Zonulin | BX | Number |
|---|---|---|---|---|
| + | + | + | neg | 0 |
| | | + | neg | 57 |
| | + | + | neg | 0 |
| | + | | neg | 3 |
| + | + | | neg | 2 |
| + | | + | neg | 10 |
| + | | | neg | 3 |
| | | | neg | 22 |
| | | | | 97 |

Thirty-three of the 68 patients from group A (48%) and 1/98 (1%) from group B showed histological findings compatible with CD (Marsh IIIa-IIIc). Of the 34 CD patients identified, 27 tested positive to all three tests, 4 tested positive to TTG and zonulin, and 3 tested positive to zonulin alone. As shown in Tables 5 and 6, the combination of AAA, TTG, and zonulin tests showed a sensitivity of 79%, a positive predictive value (PPV) of 100%, and a negative predictive value (NPV) of 100%.

TABLE 5
Group A subjects having malabsoption symptoms (n = 68)

| No CD (n = 35) | | | | CD (n = 33) | | | |
|---|---|---|---|---|---|---|---|
| AAA | tTG | Zonulin | Total | AAA | tTG | Zonulin | Total |
| + | + | + | 0 | + | + | + | 26 |
| | | + | 21 | | | + | 3 |
| | + | + | 1 | | + | + | 4 |
| | + | | 0 | | + | | 0 |
| + | + | | 0 | + | + | | 0 |
| + | | + | 2 | + | | + | 0 |
| + | | | 0 | | | | 0 |
| | | | 11 | | | | 0 |

TABLE 6
Group B subjects not having malabsoption symptoms (n = 98)

| No CD (n = 97) | | | | CD (n = 1) | | | |
|---|---|---|---|---|---|---|---|
| AAA | tTG | Zonulin | Total | AAA | tTG | Zonulin | Total |
| + | + | + | 0 | + | + | + | 1 |
| | | + | 57 | | | + | 0 |
| | + | + | 0 | | + | + | 0 |
| | + | | 3 | | + | | 0 |
| + | + | | 2 | + | + | | 0 |
| + | | + | 10 | + | | + | 0 |
| + | | | 3 | | | | 0 |
| | | | 22 | | | | 0 |

In subject experiencing malabsorptive symptoms, the combination of positive AAA, TTG, and zonulin can be used to rule in CD without the necessity to perform an EGD. This approach allows avoiding an invasive procedure in 38% of the cases of malabsorption enrolled in this study, an approach that could translate in cost saving and better patient's acceptance. The combination of negative AAA, TTG, and zonulin rules out CD without the necessity of any additional study. In these cases, an EGD could be still necessary to rule out other causes of malabsorption.

Serum zonulin measurement by sandwich enzyme-linked immunosorbent assay. Zonulin sandwich enzyme-linked immunosorbent assay (ELISA) was performed as described in El Azmar et al. Gastroenterology 123:1607-1615, 2002 with minor modifications.

Briefly, plastic microtiter plates (Costar, Cambridge, Mass.) were coated with rabbit zonulin cross-reacting anti-Zonula occludens toxin (Zot) derivative ΔG IgG antibodies (10 μg/ml in 0.1 mol/l sodium carbonate buffer, pH 9.0). These antibodies were prepared by immunizing a rabbit with ΔG using standard protocols.

After overnight incubation at 4° C., plates were washed four times in Tris buffered saline 0.05% Tween 20 (TBS-T) and blocked by incubation for 1 h at 37° C. with TBS-T. After four TBS-T washes, five ΔG serial standards (50, 25, 12.5, 6.2, 3.1, and 0 ng/ml) and patient sera samples (1:101 dilution in TBS-T) were added and incubated overnight at 4° C. After four washes with Tris buffered saline 0.2% Tween 20 buffer, plates were incubated with biotinylated anti-Zot IgG antibodies (U.S. Pat. No. 5,945,510) for 4 h at 4° C. and contacted with streptavidin-conjugated alkaline phosphatase. A color reaction was developed by using a commercial kit (ELISA amplification kit; Invitrogen). The absorbance at 495 nm was measured with a microplate auto-reader (Molecular Devices Thermomax Microplate Reader).

Auto antibodies may be detected using commercially available kits: for example, anti-(human)-transglutaminase (anti-tTG) enzyme-linked immunosorbent assay (ELISA), Scimedx Corporation, and anti-actin auto-antibodies (INOVADX, San Diego, Calif. catalog number 708785, QUANTA Lite™ Actin IgG). Alternatively, the antigen (i.e., actin or tissue transglutaminase) may be fixed to a solid support (e.g., a microtiter plate) using techniques well known in the art. The fixed antigen can be contacted with a sample to be tested for auto-antibodies. Unreacted sample may be washed off and the presence or absence of the auto-antibody may be determined by contacting the fixed antigen with an anti-human-IgG antibody (for example, a rabbit anti-human IgG). The anti-human IgG antibody may be labeled as is known in the art, for example, may be conjugated to an enzyme (i.e. peroxidase, alkaline phosphatase, luciferase, etc) and the presence of the enzyme may be detected.

While the invention has been described in detail, and with reference to specific embodiments thereof, it will be apparent to one of ordinary skill in the art that various changes and modifications can be made therein without departing from the spirit and scope thereof and such changes and modifications may be practiced within the scope of the appended claims. All patents and publications herein are incorporated by reference to the same extent as if each individual publication was specifically and individually indicated to be incorporated by reference in their entirety.

What is claimed is:

1. A method of diagnosing celiac disease in a subject, comprising:
obtaining a sample from the subject; and
determining zonulin and at least one auto-antibody in the sample, wherein the presence of zonulin and the auto-antibody is predictive of celiac disease.

2. A method according to claim 1, wherein the sample is a blood sample.

3. A method according to claim 2, further comprising:
isolating serum from the sample.

4. A method according to claim 1, comprising determining anti-actin auto-antibodies.

5. A method according to claim 1, comprising determining anti-tissue transglutaminase auto-antibodies.

6. A method according to claim 1, comprising determining serum zonulin, anti-tissue transglutaminase auto-antibodies and anti-actin auto-antibodies.

7. A method according to claim 1, wherein determining zonulin concentration comprises:
contacting the sample with a first antibody that binds to zonulin under binding conditions;
contacting the bound sample with a second antibody that binds zonulin under binding conditions; and
detecting the presence of bound second antibody.

8. A method according to claim 7, wherein at least one antibody was raised against a protein comprising a fragment of zonula occludens toxin.

9. A method according to claim 7, wherein the first antibody was raised against a protein comprising a fragment of zonula occludens toxin.

10. A method according to claim 7, wherein the first antibody was raised against a protein comprising ΔG fragment of zonula occludens toxin.

11. A method according to claim 7, wherein at least one antibody was raised against a protein comprising zonula occludens toxin.

12. A method according to claim 7, wherein the second antibody was raised against a protein comprising zonula occludens toxin.

13. A method according to claim 12, wherein the second antibody comprises a detectable moiety.

14. A method according to claim 13, wherein the detectable moiety is biotin.

15. A kit for diagnosing celiac disease, comprising:
means for detecting zonulin; and
means for detecting at least one auto-antibody.

16. A kit according to claim 15, wherein the means for detecting zonulin comprises:
a first container containing a first antibody and a second container containing a second antibody.

17. A kit according to claim 15, further comprising a container containing ΔG fragment of zonula occludens toxin.

18. A kit according to claim 16, wherein at least one antibody was raised against a protein comprising a fragment of zonula occludens toxin.

19. A kit according to claim 16, wherein the first antibody was raised against a protein comprising a fragment of zonula occludens toxin.

20. A kit according to claim 16, wherein the first antibody was raised against a protein comprising ΔG fragment of zonula occludens toxin.

21. A kit according to claim 16, wherein at least one antibody was raised against a protein comprising zonula occludens toxin.

22. A kit according to claim 16, wherein the second antibody was raised against a protein comprising zonula occludens toxin.

23. A kit according to claim 16, wherein the second antibody comprises a detectable moiety.

24. A kit according to claim 23, wherein the detectable moiety is biotin.

25. A kit according to claim 15, further comprising a container selected from the group consisting of a container containing zonulin, a container containing actin, and a container containing tissue transglutaminase.

26. A kit according to claim 15, comprising means for detecting anti-actin auto-antibodies.

27. A kit according to claim 15, comprising means for detecting anti-tissue transglutaminase auto-antibodies.

28. A method of diagnosing celiac disease in a subject, comprising:
obtaining a sample from the subject; and
determining zonulin, anti-actin auto-antibodies, and anti-tissue transglutaminase auto-antibodies, wherein the presence of zonulin, anti-actin auto-antibodies, and anti-tissue transglutaminase auto-antibodies is predictive of celiac disease.

29. A kit for diagnosing celiac disease, comprising:
means for detecting zonulin;
means for detecting anti-actin auto-antibodies; and
means for detecting anti-tissue transglutaminase auto-antibodies.
